(19) United States
(12) Patent Application Publication
(10) Pub. No.: US 2011/0262469 A1
(43) Pub. Date: Oct. 27, 2011

(54) MALARIA VACCINE BASED ON FRAGMENTS AND COMBINATION OF FRAGMENTS OF THE CS PROTEIN OF PLASMODIUM VIVAX

(75) Inventors: Socrates Herrera Valencia, Cali (CO); Myriam Arevalo-Herrara, Cali (CO); Giampietro Corradin, Epalinges (CH)

(73) Assignee: Centro Internacional De Vacunas, Cali (CO)

(21) Appl. No.: 12/444,535

(22) PCT Filed: Oct. 4, 2006
(86) PCT No.: PCT/IB2006/003263

§ 371 Date: Dec. 29, 2009

(51) Int. Cl. A61K 39/00; C07H 21/00; A61P 37/02; C12N 5/10; A61K 31/7088; C07K 14/00; C12N 15/63

(52) U.S. Cl. 424/185.1; 530/350; 536/23.1; 435/320.1; 435/325; 514/44 R

(57) ABSTRACT

The invention relates to a recombinant or synthetic polypeptide characterised in that it includes at least three consecutive repetitions of nonapeptide A N G A G X1 Q X2 X3, in which X1 is selected from D and N, X2 is selected from P and A and X2 is selected from G and A. The inventive polypeptide also preferably includes at least two (2) consecutive repetitions of sequence GDRADGQPA and in an even more preferable embodiment the polypeptide includes an amino-terminal region, a C-terminal region and/or the ptt30 fragment. The invention also relates to malaria vaccines characterised in that they include said peptides.

### FIELD OF THE INVENTION

The following described invention relates to vaccines against malaria based on B epitopes, T helpers and CD8⁺ of the circumesporozoite protein (CS protein) of P. vivax, which avoid the invasion of the parasite into the hepatic cell and its further multiplication within the same.

### BACKGROUND OF THE INVENTION

Malaria is one of the major health problems of world public health. It is estimated that every year 500 million clinic cases are produced worldwide, and that around 3 million children and pregnant women die because of that disease, each year, only in Africa. In addition to the implications which said disease has on permanent population of malaria areas, a growing amount of non-immune individuals travel each year to endemic areas and are exposed to the infection and its complications.

Africa is the most affected continent by malaria, mainly by the Plasmodium falciparum, the most virulent and responsible for 80% of the worldwide malaria. The second abundant species is the P. vivax, representing around 20% of the cases worldwide and which is significantly transmitted in the American and Asian continents. In these 2 continents the majority of the endemic regions have simultaneous transmission of Plasmodium falciparum and P. vivax. In many malaria regions, the prevalence of P. vivax is greater, and despite of not causing high mortality it produces high morbidity.

The P. vivax characterized by producing a weakening disease which causes high incapacity and presents a recurrent behavior, if the infection is not duly treated. This last characteristic represents a high risk for tourists and travelers whom in turn once infected for the first time, can develop recurrent infections without the need of being exposed again to the mosquito bite.

Having such pictorial, there is no doubt that malaria has a negative impact on social and economic development of endemic areas, to such degree, that it is estimated that the accumulated gross inner product of endemic countries for malaria has been reduced in a 50% during the last 20 years when compared to non-endemic countries.

Today, the control, managing and treatment costs are excessively high. The World Health Organization (WHO) has estimated that for 2007, the annual cost for preventing malaria only in Africa would be around US $ 2.5 billion.

The classic control methods recommended by the World Health Organization since various decades, consist in two alternatives:

1. Vector control through elimination of their breeding place, the use of repellents and small awnings, and their elimination through the use of residual action insecticides.
2. Preventive and curative treatment of the exposed or infected individuals with malaria, through the use of anti-malaria medicaments.

Despite the effort addressed to controlling this disease, faults have been evidenced in the classic control measures for malaria, due to the resistance of the parasites to the anti-malaria therapy and to the resistance to the insecticides of the Anopheles mosquito which produces an increased complexity and an increase in the cost of controlling malaria worldwide. The above facts added to factors such as deforesting, migrations and policy instability of communities of the endemic areas contribute to worsen the problem.

Given the above situation, during the last two decades, important efforts have been carried out for developing alternative strategies for controlling its transmission, within which, the vaccines have attracted the major interest due to its great potential cost-benefit.

Now, in order to carry out vaccine development against malaria, it is necessary to know the life cycle of Plasmodium, determining what is common to the 4 species of this genre (Plasmodium Spp) P. falciparum, P vivax, P. malariae and P. ovale that affect human being, and to establish which are the molecules involved in their maintenance.

It is currently known that during their life cycle, the parasite is transmitted from an infected individual to another through mosquitoes belonging to the genre of Anopheles spp, which through a bite inject the parasite in the form of a esporozoite into the blood stream of the human being. The parasites travel through the blood and reach the liver wherein they introduce into the liver cells and develop a massive multiplication phase (schizogonic cycle) which generates thousands of new parasites (merozoites) having the capacity of invading the erythrocytes, within which the parasite develops successive multiplication and reinvasion cycles into new red cells, rapidly increasing their number in the organism.

This last series of events which occur in the blood are responsible of the disease and can lead to death. Some of the parasites in the blood are sexually different as gametocytes (masculine and feminine) which when ingested by the mosquito during a new bite, carry out a fertilization process and start a new cycle (sporogonic or sexual) in the mosquito intestine, which generate new infectious sporozoites.

Within the complex life cycle of the Plasmodium, 3 distinct sites have been identified in which an anti-malaria vaccine could act on: 1) In the pre-erythrocytic phase of the cycle, that is, before the parasite enters the liver or during its development thereof; 2) during its development in the blood, that is, before the invasion of the red cells or after, during its intracellular development; and 3) during its sexual development, fertilization and development in the mosquito intestine.

The studies made up to now makes evident that the hepatic cycle of Plasmodium, has a great importance, as the parasite initiates the infection of human beings in the liver and the sporozoite inoculated by the mosquito invades starting a silent multiplication, during which the infection develops with no clinic signals present. Moreover, the results prove that in infection by P. vivax, some of the hepatic parasites can stop their development and transform in hibernated forms or hipnozoites, which can be reactivated months or years later and give rise to new malaria episodes. So, it has been considered that the total blocking of this phase with medicaments or vaccines would allow preventing the disease and the inherent risks thereof.

The natural or artificial blocking of the invasion of the parasite into the hepatocyte can be obtained through inhibition of the ligand-receptor interaction on the parasite and the host cell surfaces, or the inhibition of the development of the parasite inside thereof can be inhibited through soluble chemical mediators which prevent the multiplication process. These two methods of blocking prevent the further development of the infection and the subsequent malaria disease.

This theory has been confirmed by proving that the pre-erythrocytic phase, which starts when the sporozoites enter into the blood stream and its later invasion of the liver, can be prevented through vaccination of animals and human volunteers with attenuated sporozoites through radiation. The so human volunteers receiving the vaccine are solidly protected from the later infection with feasible sporozoites.

Unfortunately, this method of protection has been proven to have great practical type of limitations, due to the difficulty for producing the required amounts of infected mosquitoes and the impossibility of preserving breeding thereof. As a result of these impediments, a great effort has been concentrated during the last decades to identify the molecules involved in this protection, with the purpose of producing vaccines based on subunits of the parasite.

For that purpose, serum and cell from radiated sporozoite vaccine protected individuals have been used, which proved the capacity for recognizing multiple proteins of the sporozoite surface, in particular the circumsporozoite protein (CS). This molecule is used by the parasite as a ligand for the invasion to the liver and has been found in all the Plasmodium species currently studied.

These proteins have been characterized chemically and immunologically in several parasite species, including P. vivax, and it has been demonstrated that it has fragments of the molecule that interact with receptors of the hepatic cells. Also, this tests allowed the determination of the blocking of the interaction between the CS protein and the receptors of the hepatic cell, through antibodies induced through vaccination.

Based on said studies protein CS has been considered as one of the pre-erythrocytic antigens having major potential in developing a vaccine against this disease. Proteins CS possess a similar basic structure with a central region comprised by a variable number of repeated aminoacids blocks (R region) encompassing around 50% of the total protein and two flanking regions, one called Amino (N) and the other Carboxyl (C). The three regions could have vital functions for the parasite, including their function of invasion receptors.

A fragment of protein CS of P. falciparum produced through recombinant technology, has been used to apply a vaccine to human volunteers. The selected fragment comprises part of the sequence corresponding to the repetitive central region of the molecule and the totality of the carboxyl terminal region. Multiple studies have proved that this fragment in association with the soluble antigen of hepatitis B. called RTS,S/ASO2 induces protection to the vaccinated individuals against the induced infection or produced in an experimental manner with infected mosquitoes in lab or against the natural infection transmitted in malaria areas. These tests have evidenced that both adults and children of Africa can be protected between 29-34% respectively when immunized with this vaccine.

During the last decades, it has been established the complete sequence of the CS gene of P. vivax, and a series of tests have been carried out with different fragments of the protein, in order to determine the immunological most relevant domains, as well as their immunogenic capacity in test animals and human volunteers.

Using a fragment representing around 70% of the CS protein produced through recombinant technology, studies were carried out for determining the safety and immunogenicity of said fragment in a total of 30 human volunteers whom were intramuscularly inoculated with a doe between 50-400 micrograms of the aluminum hydroxyl absorbed vaccine, that the recombinant products were safe but did not have immunogenic capacity.

Among the advances reported in that sense the patent application U.S. Pat. No. 4,693,994 which discloses a synthetic peptide capable for inducing protection by antibodies against the malaria infection caused by P. vivax sporozoites, is highlighted. In said application a repeated sequence of nine aminoacids within the CS protein was described as an immune-dominant synthetic peptide. Tests carried out with these sequences highlight that even if the peptides disclosed by McCutchan and others stimulate the development of anti-CS antibodies in human beings, said peptides are not capable of inducing a significant protection.

Another application addressed to the CS protein is the patent application EP229829, which discloses the complete sequence of the P. vivax CS protein isolated from nature, characterized as a sequence of aminoacids comprising at least two repetition in tandem of the Asp-Arg-Ala-X-Gly-Gln-Pro-Ala-Gly wherein X can be Asp or Ala, preferably the application claims the protein where the tandem sequence is repeated 19 times, which is the number of times which said sequence is present in the native protein. In the same way, the document in question describes the N-terminal region, which sequence corresponds to a (MKNFILLAVS SILLVDLFPT HCGHNVDLSK AINLNGNNFN NEVDASSLGA AHVGQSASRG RGLGENPDDE EGDAKKK), the C-terminal region which sequence is (PNAKSVKEYL DKLETTVGTE WTPCSVTCGV GVRVRSRVNA ANKKPEDLTL NDLETDVCTM DKCAGIFNVV SNSLGLVILL VLALFN), and encompasses the polypeptide comprising the N-terminal and C-terminal regions separated by a sequence of non repeated aminoacids corresponding to the sequence K D G K K A E P K N P R E N K Q P R.

Even though this application marked the start of the investigations on vaccines based on the repetitive fragment of the central region of the P. vivax CS protein, the studies on efficacy of a vaccine comprising the protein with 19 repetitions of the fragment Asp-Arg-Ala-X-Gly-Gln-Pro-Ala-Gly allowed determining that the humoral and cellular immune response of the majority of the individuals whom received the vaccine was minimum.

Another relevant application in the investigation of the CS protein is the application EP0600884 which claims a P. vivax synthetic peptide containing at least one repetition of a synthetic peptide having the amino sequence Ala-Gly-Asp-Arg (AGDR), where it is expressly established that said peptide does not include the sequence of aminoacids Gly-Asp-Arg-Ala-Asp-Gly-Gln-Pro-Ala.

Addressed to the protection of other peptides derived from CS, the application EP 0392820 refers to peptides lacking of one or more repeated epitopes of the native CS protein and concentrates in N-terminal and C-terminal regions of said protein.

Even though there are several constructions of antigens derived from P. vivax proteins in the state of the art, even developed from the sequence of the P. vivax CS and the vaccines comprising thereof, none of them induce a response in vivo in humans indicating a protecting capacity against malaria. Therefore, there is the need of developing new antigens which immunogenicity is greater than that of those existing today and which response in vivo is sufficiently strong for developing a vaccine that protects against the infection with P. vivax.

### DESCRIPTION OF THE FIGURES

FIG. 1. Schematic representation of the 28 evaluated peptides, using one single code of aminoacids.

FIG. 2. Mapping the repetitive R central region of the P. vivax CS protein.

FIG. 3. Mapping the Amino (N-) and Carboxyl (C-) terminal region of the P. vivax CS protein.

FIG. 4. Recognizing T helper epitopes in the P. vivax CS protein.

FIG. 5. Identification of CD8+ epitopes derived from the P. vivax CS protein restricted to HLA-A2 molecules in malaria naturally exposed individuals.

FIG. 6. Percent of individuals with antibodies against the different domains of the P. vivax CS protein using long N, R and C peptides

FIG. 7. Immunogenicity of long peptides in BALB/c mice

FIG. 8. Immunogenicity in Aotus monkeys of a mixture of long N, C and R peptides formulated in Montanide ISA-720 and Freund adjuvants.

FIG. 9. Immunogenicity of long N, C and R peptides formulated in Montanide ISA-720 in human subjects.

### DETAILED DESCRIPTION OF THE INVENTION

In that order of ideas, the present invention focus in the development of new peptides that on their immunogenic capacities consolidate as candidate for preparing vaccines against malaria. Specifically, the disclosed peptides and vaccines in this application address to blocking the parasite hepatic phase, in which the invasion of the hepatic cell (hepatocyte) occurs through interaction of molecules (ligand) of the parasite surface and molecules (receptor) present on the hepatocyte surface. The intracellular development and multiplication can further be blocked through the action of cytokines induced by the protein in particular the gamma interferon (IFN-γ), the interleukin 6 (IL-6) and the interleukin 12 (IL-12).

The following described invention refers to vaccines against malaria based on B epitopes, T helpers and CD8+ of the protein of P. vivax circumsporozoite (CS protein), which achieve preventing the invasion of the parasite into the hepatic cell and its further multiplication within it. Said vaccines have been produced from the characterization of the CS protein and its knowledge.

The present invention constitutes a unique approach for the development of new immunologic molecules, as it is based in sequences of the P. vivax CS protein in their known form as common sequence (VK210) and in the known form as the variant (VK247).

In first place the invention is addressed to a synthetic or recombinant peptide consisting in at least three tandem repetitions of the sequence called variable or Rv, corresponding to the nona-peptide define below:

A N G A G X₁ Q X₂ X₃

Where X₁ is selected between D and N,
X₂ is selected between P and A, and
X₃ is selected between G and A.

In a concrete manner, the present invention refers to the 3Rv polypeptide corresponding to the sequence:

A N G A G X₁ Q X₂ X₃ A N G A G X₁ Q X₂ X₃ A N G
A G X₁ Q X₂ X₃

Where X₁ is selected between D and N,
X₂ is selected between P and A, and
X₃ is selected between G and A.

Preferably the polypeptide of the invention is:

| | |
|---|---|
| ANGAGNQPG ANGAGNQPG ANGAGNQPG | (SEQ ID N^{o}1) |

A second aspect of the invention provides a synthetic or recombinant peptide comprising three (3) repetitions of the peptide A N G A G X₁ Q X₂ X₃ Where X₁ is selected between D and N, X₂ is selected between P and A, and X₃ is selected between G and A, and at least two (2) repetitions of the sequence GDRADGQPA in any order.

Preferably, the peptides of the invention is the polypeptide comprising at least three repetitions of the peptide A N G A G X₁ Q X₂ X₃ followed by at least two (2) repetitions of the sequence GDRADGQPA, or the polypeptide comprising at least two (2) repetitions of the sequence GDRADGQPA followed by at least three (3) repetitions of the peptide A N G A G X₁ Q X₂ X₃. Specially the claimed invention preferably refers to proteins 3Rv3Rc and 3Rc3Rv corresponding to the sequences:

Protein 3Rv3Rc
(SEQ ID N^{o}2)
ANGAGNQPG ANGAGNQPG ANGAGNQPG GDRADGQPA GDRADGQPA
GDRADGQPA
Protein 3Rc3Rv
(SEQ ID N^{o}3)
GDRADGQPA GDRADGQPA GDRADGQPA ANGAGNQPG ANGAGNQPG
ANGAGNQPG

Another claimed invention in the present application relates to polypeptides comprising the sequence SEQ ID 1, SEQ ID 2 or SEQ ID 3, and include in their amine end the N-terminal region of PvCS corresponding to the sequence comprised between aminoacids 6-96 (90 mer) or in their carboxyl end a C-terminal peptide comprised between the aminoacid residues 301-372 (71 mer) of the P. vivax CS protein, such as the polypeptides identified as sequences SEQ ID No. 4, SEQ ID No.5, SEQ ID No. 7, SEQ ID No. 8, SEQ ID No.9, SEQ ID No.10.

In a preferred manner, the invention refers to polypeptides comprising the sequence SEQ ID 1, SEQ ID 2 or SEQ ID 3, and include in their amine end the polypeptide of the N-terminal region (90 mer) and in their carboxyl end a C-terminal peptide (71 mer) of the P. vivax CS protein, defined as the sequences SEQ ID No. 6, SEQ ID No.11, SEQ ID No. 12, described below:

Protein N+3Rv
(SEQ ID N^{o}4)
LLAVS SILLVDLFPT HCGHNVDLSK AINLNGVX1FN NVDASSLGAA
HVGQSASRGR GLGENPDDEE GDAKKKKDGK KAEPKNPREN KLKQP
ANGAGNQPG ANGAGNQPG ANGAGNQPG

where X₁ is selected between N and G

Protein 3Rv+C
(SEQ ID N^{o}5)
ANGAGNQPG ANGAGNQPG ANGAGNQPG NEGANA PNEKSVKEYL
DKVRATVGTE WTPCSVTCGV GVRVRRRVNA ANKKPEDLTL
NDLETDVCTM DKCAGIFNVV SNSLGLVILL VLA
Protein N+eRv+C
(SEQ ID N^{o}6)
LLAVS SILLVDLFPT HCGHNVDLSK AINLNGVX1FN NVDASSLGAA
HVGQSASRGR GLGENPDDEE GDAKKKKDGK KAEPKNPREN KLKQP
ANGAGNQPG ANGAGNQPG ANGAGNQPG NEGANA PNEKSVKEYL
DKVRATVGTE WTPCSVTCGV GVRVRRRVNA ANKKPEDLTL
NDLETDVCTM DKCAGIFNVV SNSLGLVILL VLA
Protein N+3Rv3Rc
(SEQ ID N^{o}7)
LLAVS SILLVDLFPT HCGHNVDLSK AINLNGVX1FN NVDASSLGAA
HVGQSASRGR GLGENPDDEE GDAKKKKDGK KAEPKNPREN KLKQP
ANGAGNQPG ANGAGNQPG ANGAGNQPG GDRADGQPA GDRADGQPA
GDRADGQPA

Where X1 is selected between N and G

Protein N+3Rc3Rv
(SEQ ID N^{o}8)
LLAVS SILLVDLFPT HCGHNVDLSK AINLNGVX1FN NVDASSLGAA
HVGQSASRGR GLGENPDDEE GDAKKKKDGK KAEPKNPREN KLKQP
GDRADGQPA GDRADGQPA GDRADGQPA ANGAGNQPG ANGAGNQPG
ANGAGNQPG

where X1 is selected between N and G.

Protein 3Rv3Rc+C
(SEQ ID N^{o}9)
ANGAGNQPG ANGAGNQPG ANGAGNQPG GDRADGQPA GDRADGQPA
GDRADGQPA NEGANA PNEKSVKEYL DKVRATVGTE WTPCSVTCGV
GVRVRRRVNA ANKKPEDLTL NDLETDVCTM DKCAGIFNVV
SNSLGLVILL VLA
Protein 3Rc3Rv+C
(SEQ ID N^{o}10)
GDRADGQPA GDRADGQPA GDRADGQPA ANGAGNQPG ANGAGNQPG
ANGAGNQPG NEGANA PNEKSVKEYL DKVRATVGTE WTPCSVTCGV
GVRVRRRVNA ANKKPEDLTL NDLETDVCTM DKCAGIFNVV
SNSLGLVILL VLA
Protein N+3Rv3Rc+C
(SEQ ID N^{o}11)
LLAVS SILLVDLFPT HCGHNVDLSK AINLNGVX1FN NVDASSLGAA
HVGQSASRGR GLGENPDDEE GDAKKKKDGK KAEPKNPREN KLKQP
ANGAGNQPG ANGAGNQPG ANGAGNQPG GDRADGQPA GDRADGQPA
GDRADGQPA NEGANA PNEKSVKEYL DKVRATVGTE WTPCSVTCGV
GVRVRRRVNA ANKKPEDLTL NDLETDVCTM DKCAGIFNVV
SNSLGLVILL VLA
Protein N+3Rc3Rv+C
(SEQ ID N^{o}12)
LLAVS SILLVDLFPT HCGHNVDLSK AINLNGVX1FN NVDASSLGAA
HVGQSASRGR GLGENPDDEE GDAKKKKDGK KAEPKNPREN KLKQP
GDRADGQPA GDRADGQPA GDRADGQPA ANGAGNQPG ANGAGNQPG
ANGAGNQPG NEGANA PNEKSVKEYL DKVRATVGTE WTPCSVTCGV
GVRVRRRVNA ANKKPEDLTL NDLETDVCTM DKCAGIFNVV
SNSLGLVILL VLA

Another alternative of the invention covered by this application refers to any of the polypeptides above-defined, which furthermore presents in the N-terminal end of the tandem repetition sequences, the leader sequence (L) corresponding to the sequence K D G K K A E P K N P R E N K L K Q P. Preferably the protein comprises said sequence corresponding to sequences SEQ ID No. 13 and SEQ ID No.14.

Protein N+L+3Rv3Rc+C
(SEQ ID N^{o}13)
LLAVS SILLVDLFPT HCGHNVDLSK AINLNGVNFN NVDASSLGAA
HVGQSASRGR GLGENPDDEE GDAKKKKDGK KAEPKNPREN KLKQP
KDGKKAEPKNPRENKLKQP ANGAGNQPG ANGAGNQPG ANGAGNQPG
GDRADGQPA GDRADGQPA GDRADGQPA NEGANA PNEKSVKEYL
DKVRATVGTE WTPCSVTCGV GVRVRRRVNA ANKKPEDLTL
NDLETDVCTM DKCAGIFNVV SNSLGLVILL VLA
Protein N+L+3Rc3Rv+C
(SEQ ID N^{o}14)
LLAVS SILLVDLFPT HCGHNVDLSK AINLNGVNFN NVDASSLGAA
HVGQSASRGR GLGENPDDEE GDAKKKKDGK KAEPKNPREN KLKQP
KDGKKAEPKNPRENKLKQP GDRADGQPA GDRADGQPA GDRADGQPA
ANGAGNQPG ANGAGNQPG ANGAGNQPG NEGANA PNEKSVKEYL
DKVRATVGTE WTPCSVTCGV GVRVRRRVNA ANKKPEDLTL
NDLETDVCTM DKCAGIFNVV SNSLGLVILL VLA

Another option of the invention, refers to the synthetic or recombinant polypeptide having at least three (3) tandem repetitions of the peptide A N G A G X₁ Q X₂ X₃ Where X₁ is selected between D and N, X₂ is selected between P and A, and X₃ is selected between G and A, and at least two (2) repetitions of the sequence GDRADGQPA, followed in its amino end by the sequence (FNNFTVSFWKRVPKVSAAHLW) of the universal epitope of T cells (ptt-30). Such is the case of proteins ptt30+3Rv3Rc (SEQ ID No.15). Another option of the invention refers to the synthetic or recombinant polypeptide having at least two (2) repetitions of the sequence GDRADGQPA and at least three (3) repetitions of the peptide A N G A G X₁ Q X₂

X₃ Where X₁ is selected between D and N, X₂ is selected between P and A, and X₃ is selected between G and A, followed in its amino end by the sequence (FNNFTVSFWKRVPKVSAAHLW) of the universal epitope of T cells (ptt-30) and ptt30+3Rv3Rc, defined as sequences SEQ ID No.16.

Protein ptt30+3Rc3Rv
(SEQ ID N^{o}16)
FNNFTVSFWKRVPKVSAAHLW GDRADGQPA GDRADGQPA
GDRADGQPA ANGAGNQPG ANGAGNQPG ANGAGNQPG

Finally, another claimed invention in the present application relates to polypeptides comprising the sequence SEQ ID No.15 or SEQ ID No.16, and include in its amino end the N-terminal region polypeptide of PvCS corresponding to the sequence comprised between amino acids 6-96 (90 mer) or in its carboxyl end a C-terminal peptide comprised between the aminoacid residues 301-372 (71 mer) of the P. vivax CS protein, such as the polypeptides identified as sequences SEQ ID No.17, SEQ ID No.18, SEQ ID No.19, SEQ ID No.20.

In a preferred manner, the invention refers to the polypeptides comprising the sequence SEQ ID 15 or SEQ ID 16, and include in their amine end the N-terminal region polypeptide (90 mer) and in their carboxyl end a C-terminal peptide (71 mer) of the P. vivax CS protein, defined as sequences SEQ ID No. 21 and SEQ ID No.22 described below:

Protein N+ptt30+3RvRc
(SEQ ID N^{o}17)
LLAVS SILLVDLFPT HCGHNVDLSK AINLNGVNFN NVDASSLGAA
HVGQSASRGR GLGENPDDEE GDAKKKKDGK KAEPKNPREN KIKQF
FNNFTVSFWKRVPKVSAAHLW ANGAGNQPG ANGAGNQPG
ANGAGNQPG GDRADGQPA GDRADGQPA GDRADGQPA
Protein N+ptt30+3RcRv
(SEQ ID N^{o}18)
LLAVS SILLVDLFPT HCGHNVDLSK AINLNGVNFN NVDASSLGAA
HVGQSASRGR GLGENPDDEE GDAKKKKDGK KAEPKNPREN KLKQP
FNNFTVSFWKRVPKVSAAFHLW GDRADGQPA GDRADGQPA
GDRADGQPA ANGAGNQPG ANGAGNQPG ANGAGNQPG
Protein ptt30+3RvRc+C
(SEQ ID N^{o}19)
FNNFTVSFWKRVPKVSAAHLW ANGAGNQPG ANGAGNQPG
ANGAGNQPG GDRADGQPA GDRADGQPA GDRADGQPA NEGANA
PNEKSVKEYL DKVRATVGTE WTPCSVTCGV GVRVRRRVNA
ANKKPEDLTL NDLETDVCTM DKCAGIFNVV SNSLGLVILL VLA
Protein ptt30+3RcRv+C
(SEQ ID N^{o}20)
FNNFTVSFWKRVPKVSAAHLW GDRADGQPA GDRADGQPA
GDRADGQPA ANGAGNQPG ANGAGNQPG ANGAGNQPG NEGANA
PNEKSVKEYL DKVRATVGTE WTPCSVTCGV GVRVRRRVNA
ANKKPEDLTL NDLETDVCTM DKCAGIFNVV SNSLGLVILL VLA
Protein N+ptt30+3RvRc+C
(SEQ ID N^{o}21)
LLAVS SILLVDLFPT HCGHNVDLSK AINLNGVNFN NVDASSLGAA
HVGQSASRGR GLGENPDDEE GDAKKKKDGK KAEPKNPREN KLKQP
FNNFTVSFWKRVPKVSAAHLW ANGAGNQPG ANGAGNQPG
ANGAGNQPG GDRADGQPA GDRADGQPA GDRADGQPA NEGANA
PNEKSVKEYL DKVRATVGTE WTPCSVTCGV GVRVRRRVNA
ANKKPEDLTL NDLETDVCTM DKCAGIFNVV SNSLGLVILL VLA
Protein N+ptt30+3RcRv+C
(SEQ ID N^{o}22)
LLAVS SILLVDLFPT HCGHNVDLSK AINLNGVNFN NVDASSLGAA
HVGQSASRGR GLGENPDDEE GDAKKKKDGK KAEPKNPREN KLKQP
FNNFTVSFWKRVPKVSAAHLW GDRADGQPA GDRADGQPA
GDRADGQPA ANGAGNQPG ANGAGNQPG ANGAGNQPG NEGANA
PNEKSVKEYL DKVRATVGTE WTPCSVTCGV GVRVRRRVNA
ANKKPEDLTL NDLETDVCTM DKCAGIFNVV SNSLGLVILL VLA

Now, it is also a part of this invention the nucleic acid sequence encoding the peptides and polypeptides of the invention previously defined. As well as the nucleic acid complementary molecules thereof (DNAc) and the variations this molecules can have in DNA by virtue of the degeneration of the genetic code.

Also, the claimed invention encompasses the expression vectors comprising the DNA or DNAc define in the above paragraph and the cells transformed by said vectors. Among the vectors used in this invention there are plasmids, phages, baculovirus and Yac, expressed in prokaryotic systems such as bacteria, and eukaryotic such as yeast, plant, mammals and insect cells.

In addition to the described inventions, the invention includes also the pharmaceutical compositions, mainly vaccines for preventing malaria comprising the peptide, the polypeptides previously precised, or vectors or cells comprising the DNA from which the peptide or polypeptides of this invention are synthesized.

Preferably, the invention refers to previously precised pharmaceutical compositions that comprise one or more adjuvant for human use, which use is widely known in vaccine formulations for powering the immune response through specific antibodies induction and/or stimulating T helper and/or cytotoxic lymphocytes.

As a complement, it is an object of the present invention the formulation of previously described immunogenic molecules, individually or combined with adjuvants or combined with other immunogenic molecules formulated in pharmaceutical compositions in order to be administered in patients who need preventing malaria infections; These molecules can be formulated as pharmaceutical compositions in the form of recombinant proteins and/or synthetic peptides formulated in different adjuvants for human use and in different proportions.

Accordingly, the pharmaceutical compositions comprising immunogenic molecules as previously defined with one or more adjuvants selected from the group comprising Montanide ISA-720, Montanide ISA-51, ASO₂ (SBAS2), AS2V, AS1B, MF59, Alum, QS-21, MPL, CpG or microcapsules. These adjuvants have been used with different antigens of Plasmodium including the P. falciparum CS protein and have proved safe and stimulating the humoral and/or cellular immune response.

In addition, it is understood that the claimed invention comprises pharmaceutical compositions comprising the immunogenic molecules previously defined and derived fragments of other state of Plasmodium or of different microorganisms and optionally, include different adjuvants for human use.

Preferably, the peptides of the invention can be combined with antigens present in the different phases of the life cycle of the parasite, despite if the antigens are annexed to the sequence during synthesis or are added to the pharmaceutical composition, such as the adhesion protein related to thrombospondine (TRAP), the linkage protein to Duffy (DBP), the merozoite surface protein (MSP-1), P25 protein and P48/45 protein of the sporogonic cycle, among others. These antigens can be used complete of fragments thereof produced as synthetic peptides, recombinant proteins or DNA.

As an illustrative manner, examples, describing in a detailed manner the methodology carried out for characterizing the P. vivax CS protein and the production of the different molecules of the invention, are given below.

### Example 1

### Identification of Segments of Interest for the Invention

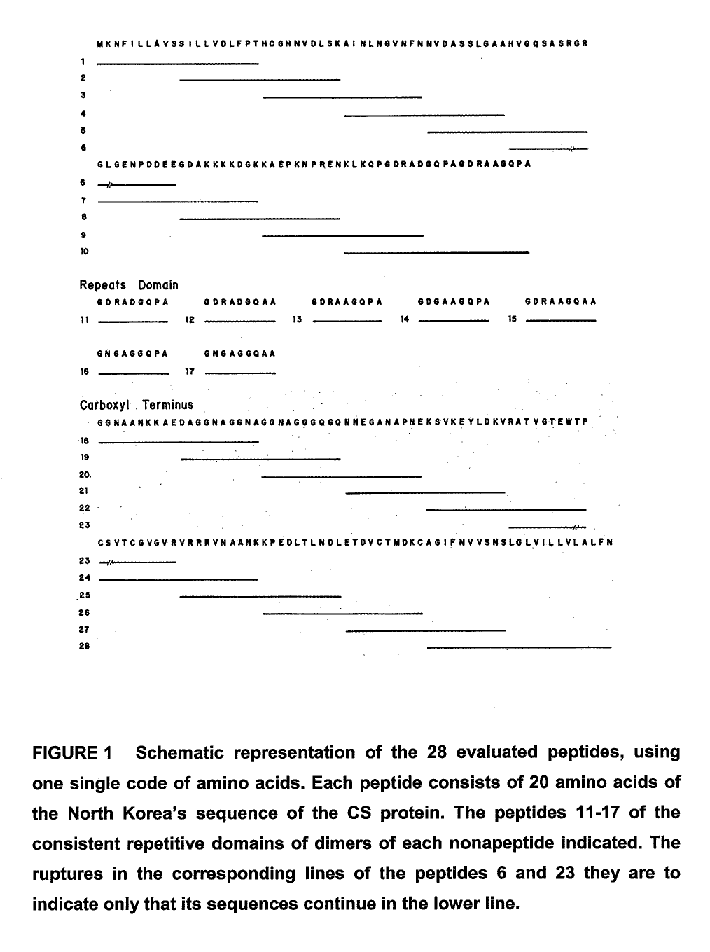

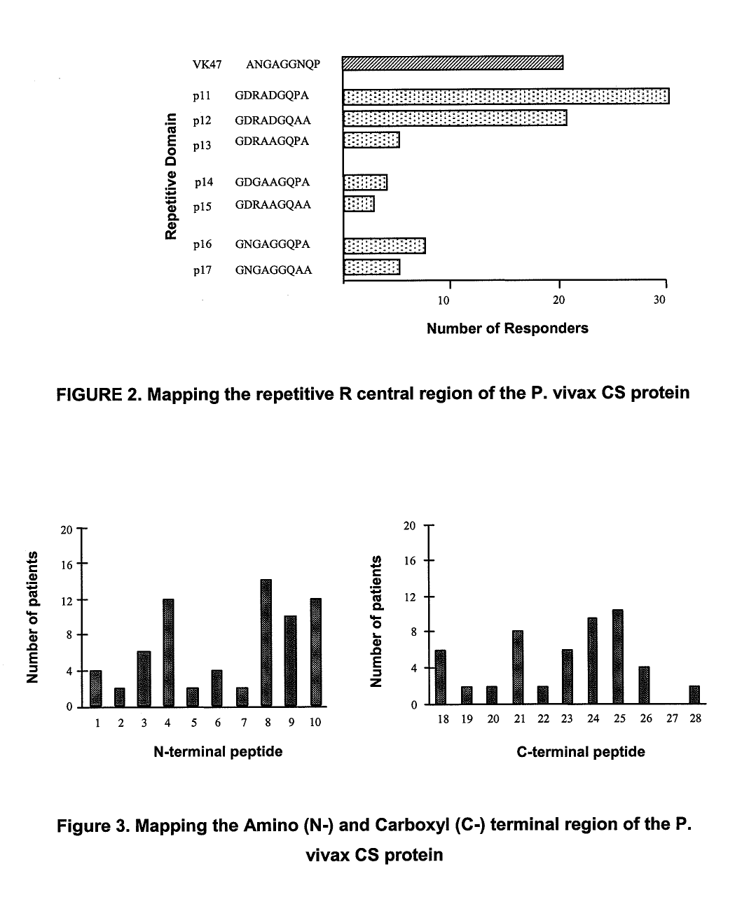

Epitopes B, T helpers and T-CD8+ are considered relevant as segments of interest to be included in a vaccine for the present invention. In order to identify B epitopes, 28 peptides from 20 overlapped residues in 10 residues each (FIG. 1) were synthesized, which were studied using serum from different individuals previously exposed to malaria and considered carriers in different grades of clinical immunity. From the 7 peptides used in the analysis of the central repetitive region, peptide P11 (GDRADGQPA or ANGAGNQPG) was recognized by the major number of individuals (FIG. 2), while from the 21 peptides used for the analysis of flanking regions N and C, peptides p8, p24 and p25 were the most frequently recognized and described as B epitopes (FIG. 3).

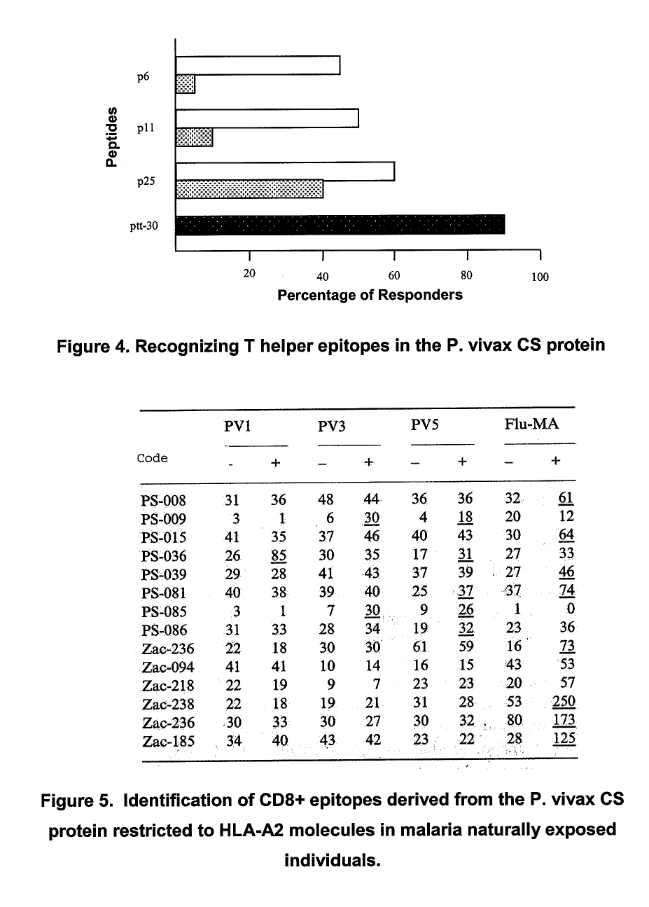

The same overlapped peptides were used in cellular proliferation tests in order to identify the T helper epitopes using peripheral blood lymphocytes (PBL) from the same individuals of endemic areas of malaria. In these studies the epitopes most frequently recognized were contained in the peptides p6, p11 and p25 and described as T helper epitopes (FIG. 4).

In addition, given the importance of the CD8+ response with the production of IFN-γ in the elimination of hepatic schizonts, a series of experiments were carried out in order to identify potential CD8+ epitopes. After selecting, through bioinformatics techniques, sequences of PvCS containing linkage motifs to antigens HLA-A*0201, a series of peptides were synthesized, which were studied using individual LSPs completing the dual requirement of being HLA-A*0201 and immune to malaria. Using this process the peptides PV-1, PV-3 and PV-5 capable of inducing the production in vitro of IFN-γ by part of the potential CD8+ lymphocytes which are cytotoxicity inductors shown in FIG. 5, were identified.

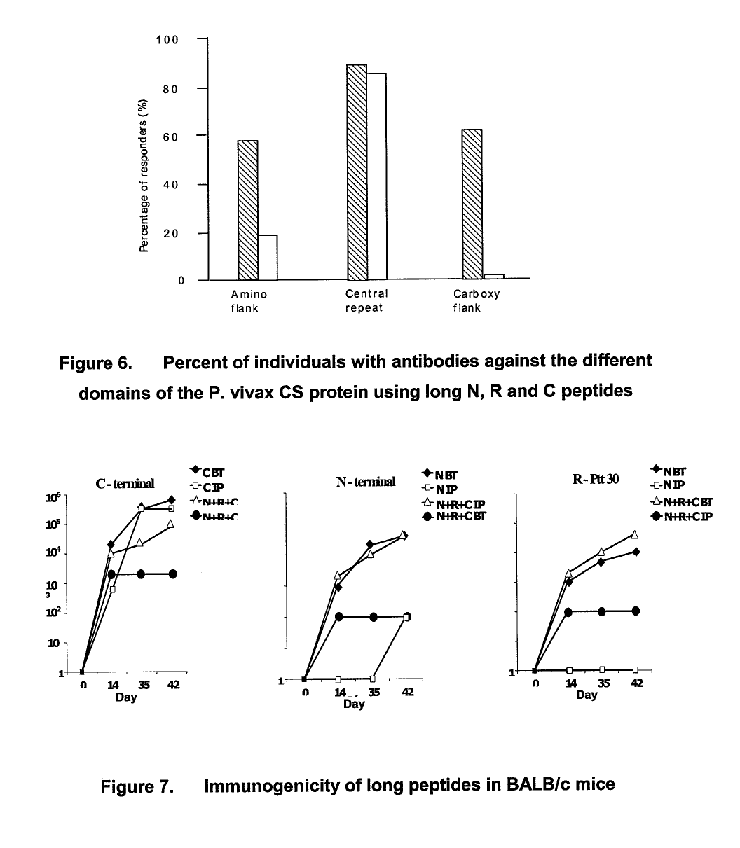

Based in the previous location of epitopes, long peptides (N, R and C) were produced, containing the different identified epitopes, and were analyzed using serum samples (n=121) from adults of the endemic regions. The three (39 regions of the protein were recognized by a significant number of individuals (N=59%, R=88%, C=63%). (FIG. 6)

It is known that the central region of CS protein can be present in nature with different sequences named type I or a common sequence (VK210-Rc) and type II or variant sequence (VK247-Rv). In the studied region, the antibodies were mainly addressed against the derived sequence type VK210 (75%), while a lesser number of individuals (20%) showed antibodies against sequence type VK247. Furthermore, antibodies addressed against the minimum epitope AGDR derived from VK210 or Re were found in 66% of the individuals from the same region. The existence of high and frequent titles of antibodies against type VK210 in the studied population, is associated with a minor prevalence of parasites with this type of sequence in the studied endemic areas and viceversa, which leaded to the proposal that the stimulation of high antibody titles through vaccination with variant VK247, would have a protecting effect against the parasite.

In addition the studies carried out for determining what type of sequence was present in the sporozoites found in infected mosquitoes in nature proved that 90% of them are recognized by monoclonal antibodies against the variant sequence (VK247). This result was confirmed by sequencing CS genes when 24 of 25 different nature isolated parasite samples were found in endemic areas bearing the VK247 sequence.

Based on the exposed results different R peptides were created which incorporated tandem repetitions of derived VK210 epitopes, derived VK247 epitopes and mixtures of those epitopes alone or in combination with N and C. These polypeptides were analyzed and the obtained results are shown below.

### Example 2

### Pre-Clinic Essays of Immunogenicity in Mice

BALB mice were used to determine the immunogenicity of the peptides (N, R, C) administered by intraperitoneal (IP) and subcutaneous (SC) ways both individually and in combined form. The peptides were formulated in Freund adjuvant. The measure of the antibodies response against each one of the peptides, was carried out through the ELISA technique during several weeks after immunization. The immunization of the mice induced a vigorous antibodies response (1:80,000-1:1,000,000), in particular against N and R peptides. (See FIG. 7).

### Example 3

### Pre-Clinic Essays on Immunogenicity in Non-Human Primates

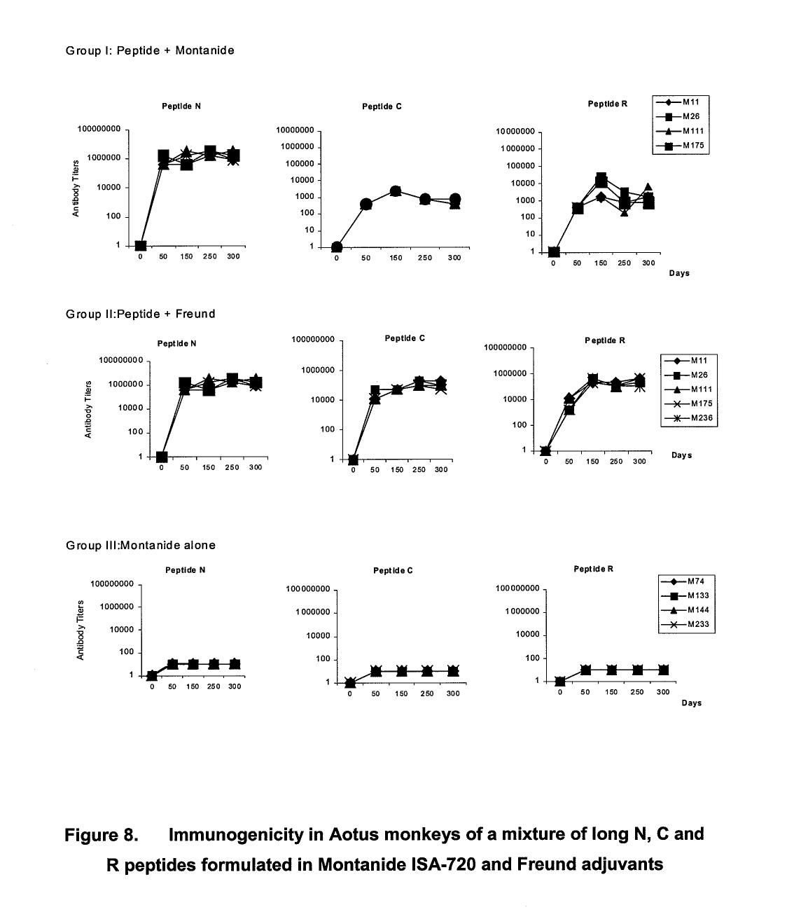

The immunogenicity of combinations of the same long peptides was studied in Aotus lemurimus monkeys, which received 3 dose of 100 μg of each of the peptides N, R and C formulated in the Montanide ISA 720 adjuvants and in the complete and incomplete Freund adjuvants. The immunizations unchained high antibodies titles against corresponding peptides N, R and C and the antibodies recognized the native protein of the parasite in immunofluorescence tests. (FIG. 8)

### Example 4

### Clinic Essays of Phase I in Humans

The safety, tolerance and immunogenicity of long peptides were studied in phase I clinic essays in young adult individuals with no malaria background, under Good Clinical Practices norms (GCP). In a first clinical test 69 volunteers were immunized randomly distributed in groups of 7 each which were immunized with one of the three (3) peptides (N, R or C) with stepping dose of the peptides (10, 30 and 100 micrograms/dose) formulated in the montanide ISA 720 adjuvant and applied through intramuscular way in the deltoid region. The vaccination scheme consisted in immunizations administered in months 0, 2 and 6. A group of control individuals were vaccinated with salt solution formulated in the same adjuvant.

None of the individuals showed serious adverse events and the vaccination scheme with each peptide was satisfactory completed at least by 22 of 23 volunteers of each peptide. The short duration pain (<48 hours) in the injection site was the most frequent symptom. In addition, a small edema showed in around 20% of vaccinated, which resolved prior to 48 hours. (Table 1).

TABLE 1
Most frequent clinical Symptoms and findings during follow up,
in the immunized volunteers with N, C and R long peptides.

| # | | | Local | | a- | no |
|---|---|---|---|---|---|---|
| Dose | Eritema <5 cm | Pain <48 h | edema | Prurite | sleep | symptom |
| 1 | 0 | 20 | 1 | 1 | 0 | 1 |
| 2 | 0 | 22 | 7 | 1 | 0 | 2 |
| 3 | 0 | 14 | 6 | 1 | 0 | 0 |

The paraclinical studies did not showed alterations related to the vaccine in any of the cases.

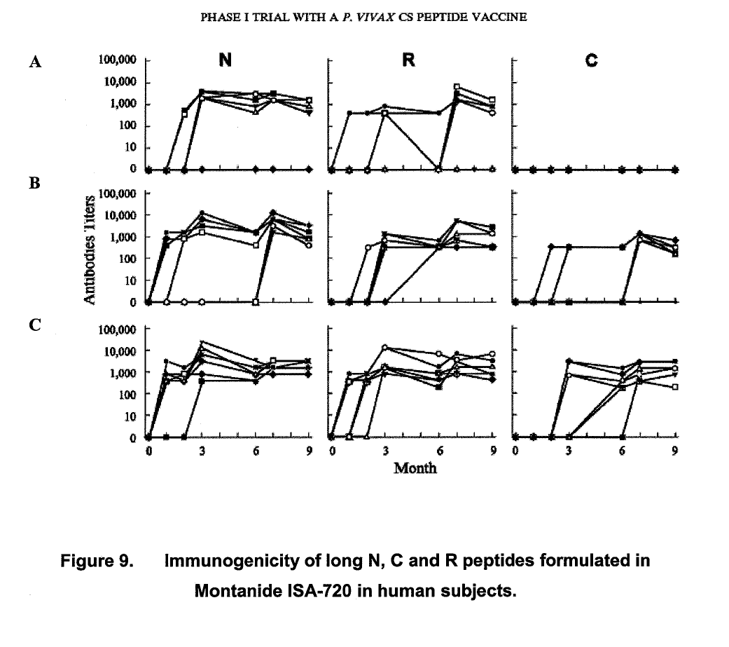

The humoral immune response was determined by the ELISA method against its immunogen and by immunofluorescence (IFAT) against the native CS protein. Using as antigen the derived synthetic peptides of the P. vivax CS, an immediate increase of antibody titles was proven in the majority of the cases. These antibodies were maintained high during the follow up period thereof and recognized the native protein expressed in the sporoziot surface (FIG. 9).

In a second clinical assay, safety, tolerance and immunogenicity of mixtures of the same long synthetic peptides (N, R and C) formulated in Montanide ISA 720 and ISA 51 were examined in 40 volunteers with no previous experience with malaria. Two of the groups were immunized with the mixture of the 3 peptides (N+R+C) with dose of 50 and 100 micrograms formulated in two adjuvants, Montanide ISA-720 and ISA-51, respectively. The vaccination scheme consisted in immunizations administered in months 0, 2 and 4 applied through intramuscular way in the deltoid region. Two groups were immunized with the adjuvants and without the peptide mixture and one group was immunized only with salt solution.

After the immunization, the volunteers were kept under observation during a period of 5 months counted from the first immunization, and was found as in the first clinical assay, none of the volunteers experimented serious adverse events, directly related to the vaccine Table 2, and the vaccine was well tolerated. Again, there was a slight, low duration pain in the puncture site (48 hours), along with local induration, edema and local erythema. There were no differences observed in the symptoms or signs related to the number of dose or the peptide used. All the lab tests remained with normal values during the period of study.

TABLE 2
Most frequent clinical Symptoms and findings during
follow up of the immunized volunteers with the mixture
of the peptides formulated in the two adjuvants.

| # | | Local | Local | | Local | no |
|---|---|---|---|---|---|---|
| Dose | Pain <48 h | induration | edema | Erythema | Heat | symptom |
| 1 | 17 | 4 | 1 | 2 | 1 | 0 |
| 2 | 16 | 1 | 1 | 0 | 0 | 0 |
| 3 | 7 | 2 | 2 | 0 | 1 | 0 |

6 evaluations were made for determining the humoral immune response using the same methods of the above assay. The analysis confirmed the capacity of the 3 peptides to stimulate the specific antibodies production having recognition of the native protein in the parasite. Also they stimulated the production of IFN-γ, and there was no antagonism or antigenic synergism observed in the formulations.

1. A recombinant or synthetic polypeptide comprising at least 3 repetitions followed by the nona-peptide:
A N G A G X₁ Q X₂ X₃
Wherein X₁ is selected from D and N,
X₂ is selected from P and A, and
X₃ is selected from G and A, and
at least two (2) repetitions followed by GDRADGQPA.

2. The recombinant or synthetic polypeptide according to claim 1, wherein the number of repetitions of the sequence GDRADGQPA is three.

3. The recombinant or synthetic peptide according to claim 2, wherein the three copies of the sequence GDRADGQPA are in C-terminal end of the peptide.

4. The recombinant or synthetic peptide according to claim 3, wherein comprising the amino acid sequence identified as SEQ ID No. 2.

5. The recombinant or synthetic peptide according to claim 2, wherein the three copies of the sequence GDRADGQPA are in N-terminal end of the peptide.

6. The recombinant or synthetic peptide according to claim 5, wherein comprising the amino acid sequence identified as SEQ ID No. 3.

7. The recombinant or synthetic peptide according to claim 1, wherein comprising in its N-terminal end, the sequence LLAVS SILLVDLFPT HCGHNVDLSK AINLNGVNFN NVDASSLGAA HVGQSASRGR GLGENPDDEE GDAKKKKDGK KAEPKNPREN KLKQP.

8. The recombinant or synthetic peptide according to claim 7, wherein comprising a sequence selected from the group consisting of SEQ ID No.4, SEQ ID No.7 and SEQ ID No.8.

9. The recombinant or synthetic peptide according to claim 1, wherein comprising in its C-terminal end the sequence NEGANA PNEKSVKEYL DKVRATVGTE WTPCSVTCGV GVRVRRRVNA ANKKPEDLTL NDLETDVCTM DKCAGIFNVV SNSLGLVILL VLA.

10. The recombinant or synthetic peptide according to claim 9, wherein comprising a sequence selected from the group consisting of SEQ ID No.5, SEQ ID No.9, SEQ ID No.10, SEQ ID No.11 and SEQ ID No.12.

11. The recombinant or synthetic peptide according to claim 1, wherein comprising in the amino end of the tandem repetition sequences, a leader sequence (L) corresponding to the sequence K D G K K A E P K N P R E N K L K Q P.

12. The recombinant or synthetic peptide according to claim 11, wherein comprising any of the sequences SEQ ID No.13, SEQ ID No.14.

13. The recombinant or synthetic peptide according to claim 1, wherein comprising in the N-terminal end of the tandem repetition sequences, the sequence FNNFTVSFWKRVPKVSAAHLW of the universal epitope of T-cells (ptt-30) derived from the tetanus toxin.

14. The recombinant or synthetic peptide according to claim 13, wherein comprising a sequence selected from the group consisting of SEQ ID No.15, SEQ ID No.16, SEQ ID No.17, SEQ ID No.18, SEQ ID No.19, SEQ ID No.20, SEQ ID No.21, SEQ ID No.22, SEQ ID No.23 or SEQ ID No.24.

15. A nucleic acid molecule characterized in that the nucleic acid encodes any of the polypeptides according to claim 1.

16. The nucleic acid according to claim 15 wherein it is a DNA, RNA or cDNA molecule.

17. An expression vector wherein it comprises the nucleic acid molecule of claim 15.

18. The expression vector according to claim 17 wherein it is a plasmid or a phage.

19. A recombinant cell wherein it comprises the expression vector of claim 17.

20. A pharmaceutical composition for malaria prevention wherein it comprises the synthetic or recombinant peptide according to claim 1, a nucleic acid molecule encoding the synthetic or recombinant peptide, or an expression vector comprising a nucleic acid molecule encoding the synthetic or recombinant peptide.

21. A vaccine for malaria prevention comprising the synthetic or recombinant peptide according to claim 1, a nucleic acid molecule encoding the synthetic or recombinant peptide, or an expression vector comprising a nucleic acid molecule encoding the synthetic or recombinant peptide.

22. The vaccine according to claim 21 further comprising one or more adjuvants for human use.

23. The vaccine according to claim 21 further comprising immunogenic molecules selected from the group consisting of Montanide ISA-720, Montanide ISA-51, ASO2 (SBAS2), AS2V, AS1B, MF59, Alum, QS-2, MPL, CpG or microcapsules.

24. The vaccine according to claim 21 further comprising fragments derived from other stages of Plasmodium or from different microorganisms.

25. The vaccine according to claim 21, wherein comprising antigens present in the various phases of the parasite life cycle, said antigens are selected from the group consisting in the adhesion protein related to thrombospondine (TRAP), the Duffy bound protein (DBP), the surface protein of merozoite (MSP-1), the protein P25 and protein P48/45, among others. These antigens can be used complete or fragments thereof produced as synthetic peptides, recombinant proteins or DNA.
